(19) United States
(12) Patent Application Publication
(10) Pub. No.: US 2004/0044316 A1
(43) Pub. Date: Mar. 4, 2004

(54) Syringe for sequential delivery of different fluids

(76) Inventor: Christian John Greenfield, Oxnard, CA (US)

Correspondence Address: Michael A. O'Neil, Michael A. O'Neil, P.C., Suite 820, 5949 Sherry Lane, Dallas, TX, 75225, US

(21) Appl. No.: 10/231,827

(22) Filed: Aug. 30, 2002

(51) Int. Cl. A61M 5/00

(52) U.S. Cl. 604/191

(57) ABSTRACT

A syringe for sequentially administering different fluids includes a floating piston which separates the syringe into a first portion for receiving a first fluid to be administered and a second portion for receiving a second fluid to be administered. The floating piston includes a fluid tight seal which initially separates the first and second portions of the syringe and a piercing needle which penetrates the fluid tight seal to connect the second portion of the syringe to the syringe needle after the first fluid has been discharged from the syringe.

### TECHNICAL FIELD

This invention relates generally to the sequential administration of different fluids, for example, the administration of a medication followed immediately by the administration of a normal saline solution, and more particularly to an improved syringe design which assures complete delivery of the first fluid followed immediately by complete delivery of the second fluid.

### BACKGROUND AND SUMMARY OF THE INVENTION

Various medical procedures require the sequential administration of different fluids. For example, administration of the cardiac drug known as Adenosine must be followed immediately by the administration of a normal saline solution in order to get the drug into circulation rapidly. Heretofore the sequential administration of different fluids has been accomplished utilizing two syringes, one loaded with the first fluid to be administered and the other loaded with the second fluid to be administered. The two needle system is not only awkward but also increases the risk that medical personnel will suffer inadvertent needlesticks.

A previous attempt to solve the foregoing problem is shown and described in published U.S. patent application US 2002-0035351-A1. Referring to FIG. 1 of the present application, the device disclosed in the published application comprises a single barrel double chamber syringe 10 having a barrel 1 and a plunger 2 which is utilized in the conventional manner to force fluids contained within the barrel 1 outwardly through a hollow needle 8. This is accomplished by means of a push stopper 3 secured to the plunger 2 and made of a pliable material to maintain a fluid tight seal around its outer periphery. A hollow floating plunger 5 situated within the barrel 1 is likewise comprised of a pliable material to maintain a fluid tight seal around its outer periphery. The floating plunger 5 divides the barrel 1 into a first compartment 4 and a second compartment 6.

The hollow needle 8 is secured in a hub 9 situated at the opposite end of the barrel 1 from the plunger 2. The hollow needle 8 comprises a first component extending outwardly from the barrel 1 and the hub 9 and a piercing component extending from the hub 9 into the second chamber 6 of the barrel 1. The hollow needle 8 is provided with a porthole 7 to assure full delivery of fluid contained within the second chamber 6.

In the operation of the syringe 10 a first fluid is loaded into the second chamber 6 and a second fluid is loaded into the first chamber 4. As the push stopper 3 is forced downwardly (FIG. 1) into the barrel 1 under the action of the plunger 2 the fluid within the first chamber 4 and the floating plunger 5 function to force the first fluid outwardly from the second chamber 6 through the hollow needle 8. As the floating plunger 5 moves downwardly it eventually engages the piercing component of the hollow needle 8 which begins to penetrate the floating chamber 5. Meanwhile, the remainder of the first fluid is forced out of the second chamber 6 through the porthole 7 and the hollow needle 8.

Continued downward movement of the push stopper 3 under the action of a plunger 2 causes the piercing component of the hollow needle 8 to fully pierce the floating plunger 5 thereby allowing the second fluid to flow outwardly from the first chamber 4 through the hollow needle 8. Movement of the push stopper 3 toward the hollow needle 8 under the action of the plunger 2 continues until all of the second fluid has been discharged from the syringe 10 through the hollow needle 8.

While eliminating the problems inherent in sequentially administering two different fluids utilizing two syringes, the device shown and described in published application US-2002-0035351-A1 involves different problems. First, the hollow needle through which the different fluids are sequentially administered must be provided with a porthole. As will be appreciated by those skilled in the art, forming a porthole in a hollow needle of the type utilized in medical syringes involves difficult and expensive manufacturing steps. Second, the axial dimensions of the hollow needle utilized in the device disclosed in the published application must be very accurately controlled. Thus, if the piercing component of the needle is too short the floating plunger will not be properly penetrated thereby preventing proper administration of the second fluid. Conversely, if the piercing component of the hollow needle is too long complete delivery of the first fluid cannot be assured.

The present invention comprises an improved syringe design which overcomes the foregoing and other problems that have long since characterized the prior art. In accordance with the broader aspects of the invention an otherwise conventional syringe is provided with a floating piston comprising upper and lower portions. The lower portion may comprise a spongy material encased in a flexible shell and having a piercing needle mounted therein. Alternatively, the lower portion may comprise a flexible shell filled with a suitable gas such as air. The upper portion comprises a seal formed from a non-coring elastomeric material.

In the practice of the invention a first fluid to be administered is loaded into the barrel of the syringe below the floating piston. A second fluid to be delivered is loaded into the barrel of the syringe above the floating piston. As the plunger of the syringe is moved into the barrel the first fluid is forced outwardly through the needle of the syringe under the action of the second fluid and the floating piston. The floating piston eventually engages the discharge end of the barrel thereby forcing the entirety of the first fluid outwardly through the needle. Continued movement of the plunger of the syringe causes the penetrating needle of the floating piston to penetrate the seal comprising the upper portion of the floating piston. At this point the second fluid is connected in fluid communication with the syringe needle through the penetrating needle of the floating piston. Further inward movement of the plunger of the syringe forces all of the second fluid outwardly through the penetrating needle of the floating piston and the syringe needle.

### BRIEF DESCRIPTION OF THE DRAWINGS

A more complete understanding of the invention may be had by reference to the following Detailed Description when taken in conjunction with the accompanying Drawings, wherein:

FIG. 1 is a longitudinal sectional view of a prior art single barrel double chamber syringe;

FIG. 2 is a longitudinal sectional view of the syringe of the present invention showing the syringe in a first stage of its operation;

FIG. 3 is an enlargement of a portion of FIG. 2;

FIG. 4 is a view similar to FIG. 2 showing the syringe at a later stage in its operation;

FIG. 5 is a view similar to FIG. 2 showing a syringe of the present invention at a still later stage in its operation; and

FIG. 6 is an illustration similar to FIG. 2 showing a syringe of the present invention at the completion of its operation.

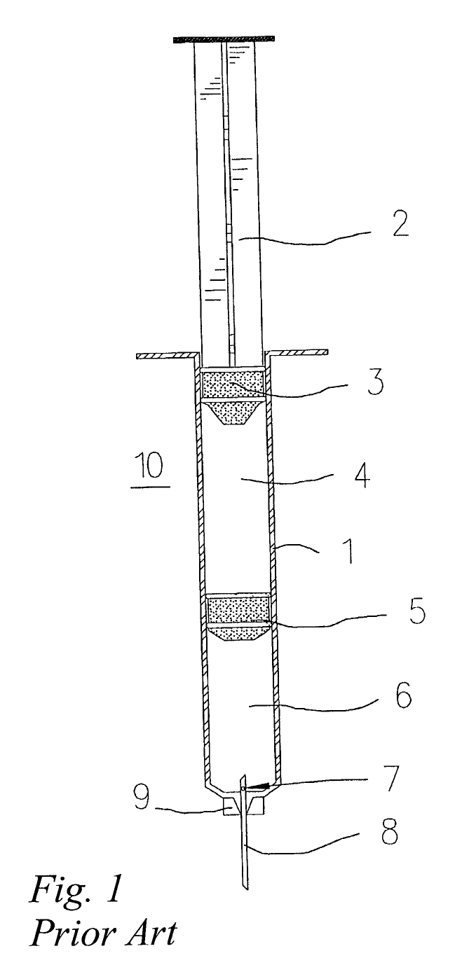

### DETAILED DESCRIPTION

Referring now to the Drawings, and particularly to FIG. 2 thereof, there is shown a syringe 20 incorporating the present invention. In many respects the syringe 20 is conventional in construction and operation. Thus, the syringe 20 includes a barrel 22 which receives fluid to be administered. A hollow needle 24 is secured to one end of the barrel 22 by a hub 26 and is coupled in fluid communication with the interior of the barrel 22. The end of the barrel 22 remote from the needle 24 may be provided with a radially extending flange 28 which is typically engaged by the fingers of an individual operating the syringe 20.

The syringe 20 further includes a plunger 30 adapted for axial movement within the barrel 22. The plunger 30 extends to a piston 32 which forms a fluid tight seal with the interior surface of the barrel 22. The end of the plunger 30 remote from the piston 32 may be provided with a plate 34 which is typically engaged by the thumb of an individual operating the syringe 20.

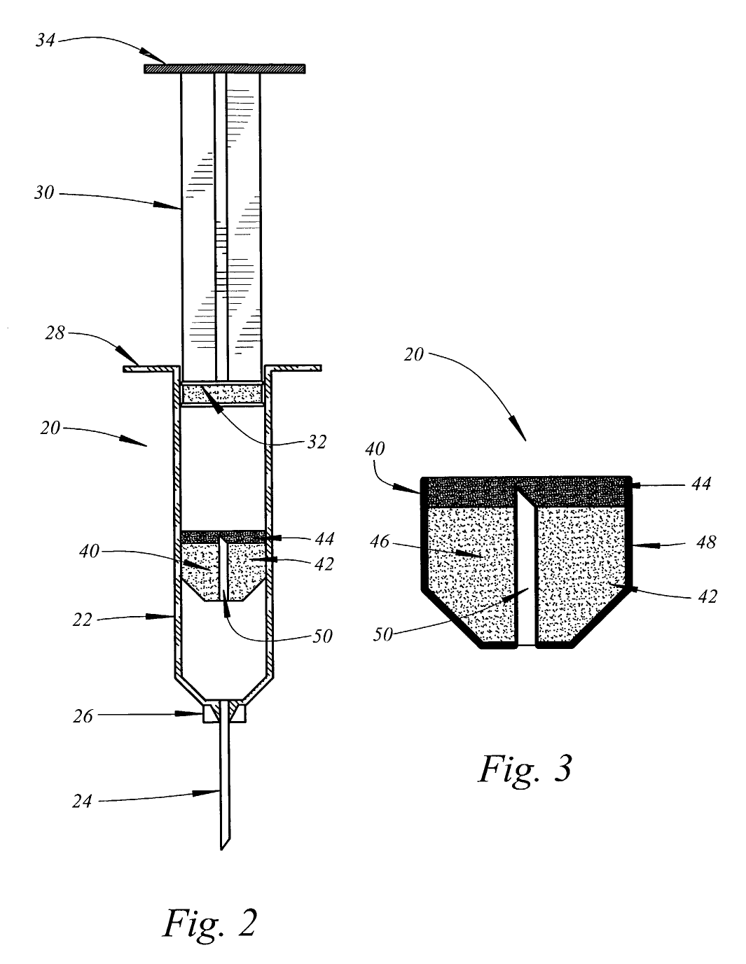

Referring to FIGS. 2 and 3, the syringe 20 differs from conventional syringes in that it is provided with a floating piston 40. The floating piston 40 includes a lower portion 42 and an upper portion 44.

Referring particularly to FIG. 3, the lower portion 42 of the floating piston 40 comprises a body 46 formed from a resilient material which may be spongy in nature. The body 46 is surrounded by a flexible layer 48 formed from a material approved for medical applications, for example, silicone. Alternatively, the lower portion 42 may comprise a flexible shell filled with a suitable gas such as air. The lower portion 42 of the floating piston 40 further comprises a piercing needle 50. The piercing needle 50 is mounted and supported by the body 46 of the lower portion 42 of the floating piston 40.

The piercing needle 50 is hollow throughout its length and is preferably either equal to or greater in diameter than the needle 24 of the syringe 20. The piercing needle 50 may be provided with an enlarged portion at the end thereof facing the needle 24 of the syringe 20 in order assure fluid communication between the piercing needle 50 and the syringe needle 24.

The upper portion 44 of the floating piston 40 comprises a non-coring elastomeric material. In use, the upper portion 44 forms a fluid tight seal with the interior surface of the barrel 22 of the syringe 20. As will be appreciated by those skilled in the art, the function of the upper portion 42 of the floating piston 40 is to divide barrel 22 of the syringe 20 into upper and lower chambers and to initially maintain a fluid-tight barrier therebetween.

Operation of the syringe 20 of the present invention is illustrated in FIGS. 2, 4, 5, and 6. Referring first to FIG. 2, a first fluid to be administered is loaded into the portion of the barrel 22 of the syringe 20 situated below the floating piston 40. A second fluid to be administered immediately following completion of the delivery of the first fluid is loaded into the portion of the barrel 22 of the syringe 20 situated above the floating piston 40. The floating piston 40, and particularly the upper portion 42 thereof, comprises a fluid tight seal which maintains separation between the two fluids.

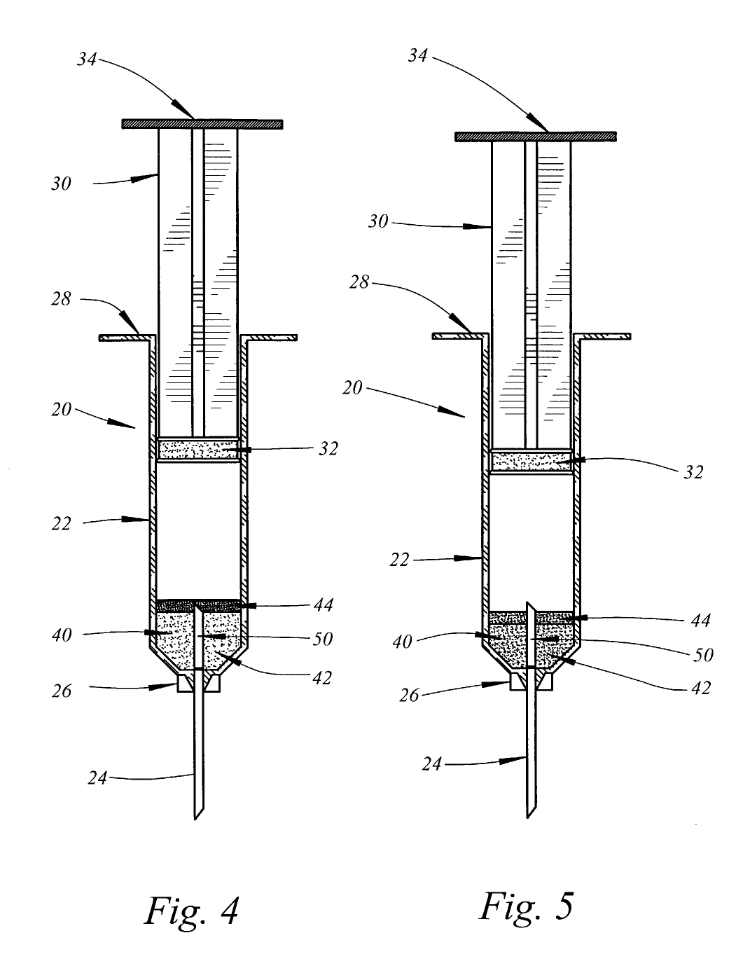

Referring next to FIG. 4, administration of the first fluid is accomplished by moving the plunger 30 inwardly, that is, from the position illustrated in FIG. 2 toward the position illustrated in FIG. 4. As the plunger 30 is moved inwardly, the floating piston 40 and the second fluid positioned within the barrel 22 of the syringe 20 betweeen the floating piston 40 and the plunger 30 function to force the first fluid out of the barrel 22 through the syringe needle 24. FIG. 4 illustrates the floating piston 40 bottomed out in the barrel 22 of the syringe 20 with all of the first fluid having been delivered through the syringe needle 24.

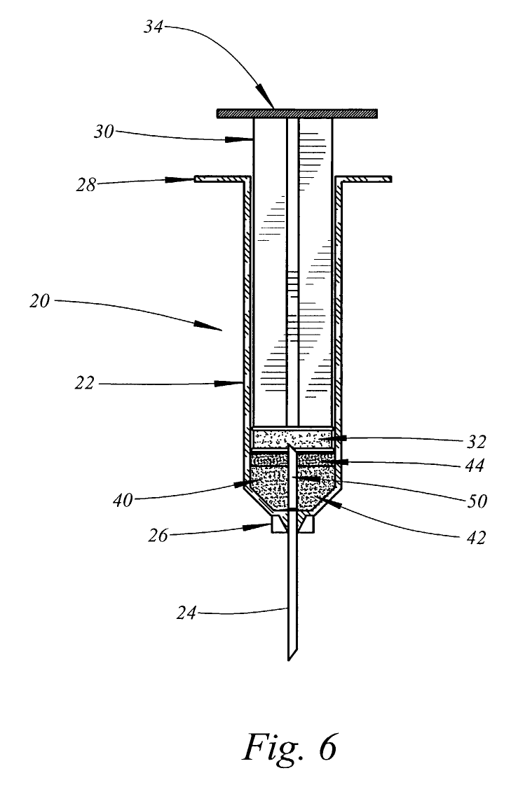

Referring next to FIG. 5, further inward movement of the piston 30 compresses the resilient material 46 and/or the gas comprising the lower portion 42 of the floating piston 40 thereby causing the piercing needle 50 to penetrate the upper portion 44 of the floating piston 40. In this manner the second fluid to be administered, which is situated between the floating piston 40 and the plunger 30, is connected in fluid communication with the syringe needle 24 through the hollow interior of the piercing needle 50. Further inward movement of the plunger 30 forces the second fluid to be administered out of the barrel 22 of the syringe 20 through the piercing needle 50 and the syringe needle 24. This action continues until the piston 32 of the plunger 30 bottoms out as illustrated in FIG. 6. At this point the syringe 20 is typically disposed of in accordance with approved syringe disposal techniques.

It will therefore be understood that the present invention comprises a syringe for sequentially administering different fluids which overcomes the problems that have characterized the prior art. In particular, manufacture of the syringe of the present invention involves the addition of a unique floating piston to an otherwise conventional syringe. Manufacture of the components parts of the syringe of the present invention does not involve complicated and expensive manufacturing techniques, nor does it involve precise control over the dimensions of the component parts of the device.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A syringe for sequentially administering different fluids comprising:
a syringe body having a hollow interior for receiving the fluids to be administered;
a syringe needle mounted at one end of the syringe body for administering the fluids therefrom;
a plunger mounted within the interior of the syringe body for movement therein toward the end of the syringe body comprising the syringe needle to force fluids outwardly from the interior of the syringe body through the syringe needle;
the plunger further comprising a piston forming a fluid tight seal with the interior of the syringe body;
a floating piston slidably supported within the hollow interior of the syringe body and forming a fluid tight seal with the syringe body which divides the syringe body into a first portion for receiving the first fluid to be administered and a second portion for receiving the second fluid to be administered; and
the floating piston further comprising means for connecting the second fluid to be administered in fluid communication with the syringe needle after all of the first fluid to be administered has been discharged therethrough.

2. The syringe according to claim 1 wherein the floating piston is further characterized by an upper portion forming a fluid tight seal with the interior of the syringe body, a lower portion comprising resilient means, and a piercing needle mounted in and supported by the resilient lower portion and having a hollow interior.

3. The syringe according to claim 2 wherein the resilient means of the lower portion comprises a spongy material.

4. The syringe according to claim 2 wherein the resilient means of the lower portion comprises a gas.

5. The syringe according to claim 2 wherein the first portion of the floating piston comprises a non-coring elastomeric material.

6. The syringe according to claim 2 wherein the lower portion of the floating piston comprises a spongy material having the piercing needle mounted therein and a resilient coating surrounding the spongy material.

7. The syringe according to claim 1 wherein the syringe needle is characterized by a predetermined diameter, and wherein the piercing needle of the floating piston is characterized by a diameter equal to or greater than the diameter of the syringe needle.

8. For use in conjunction with a syringe of the type comprising a syringe body having a hollow interior, a hollow syringe needle mounted at one end of the syringe body and coupled in fluid communication with the interior thereof, and a plunger mounted for movement within the syringe body toward the syringe needle to force fluids contained within the syringe body outwardly through the syringe needle, the plunger further comprising a piston forming a fluid tight seal with the interior of the syringe body, a floating piston adapting the syringe for the sequential administration of different fluids comprising:
means forming a fluid tight seal with the interior of the syringe body which divides the syringe body into a first portion for receiving a first fluid to be administered and a second portion for receiving a second fluid to be administered; and
means for connecting the second portion of the syringe body in fluid communication with the syringe needle after the first fluid to be administered has been discharged from the syringe body through the syringe needle under the action of the plunger.

9. The syringe according to claim 6 wherein the floating piston is further characterized by an upper portion forming a fluid tight seal with the interior of the syringe body, a lower portion comprising resilient means, and a piercing needle mounted in and supported by the resilient lower portion and having a hollow interior.

10. The syringe according to claim 9 wherein the resilient means of the lower portion comprises a spongy material.

11. The syringe according to claim 9 wherein the resilient means of the lower portion comprises a gas.

12. The syringe according to claim 9 wherein the first portion of the floating piston comprises a non-coring elastomeric material.

13. The syringe according to claim 9 wherein the lower portion of the floating piston comprises a spongy material having the piercing needle mounted therein and a resilient coating surrounding the spongy material.

14. The syringe according to claim 8 wherein the syringe needle is characterized by a predetermined diameter, and wherein the piercing needle of the floating piston is characterized by a diameter equal to or greater than the diameter of the syringe needle.

15. For use in conjunction with a syringe of the type having a cylindrical barrel, a floating piston including a fluid tight seal which initially separates first and second portions of the syringe barrel and a piercing needle which penetrates the fluid tight seal to connect the first and second portions of the syringe barrel in fluid communication.
